(19) United States
(12) Patent Application Publication
(10) Pub. No.: US 2021/0219875 A1
(43) Pub. Date: Jul. 22, 2021

(54) SYSTEM AND METHOD FOR ASSESSING STRENGTH USING WEARABLE SENSORS

(71) Applicant: KONINKLIJKE PHILIPS N.V., Eindhoven (NL)

(72) Inventors: Mladen MILOSEVIC, Stoneham, MA (US); Saman PARVANEH, Danvers, MA (US); Amir ABDOLAHI, Malden, MA (US); Roberto BUIZZA, Malden, MA (US)

(21) Appl. No.: 17/150,094

(22) Filed: Jan. 15, 2021

Related U.S. Application Data
(60) Provisional application No. 62/963,737, filed on Jan. 21, 2020.

(51) Int. Cl. A61B 5/11; A61B 5/00

(52) U.S. Cl. CPC A61B 5/1124; A61B 5/486; A61B 5/1122; A61B 5/681

(57) ABSTRACT

A strength assessment generator includes at least one sensor configured to detect movement data and a processor configured to generate a strength assessment based on the movement data detected by the at least one sensor. The at least one sensor is coupled to a body part of a person, and the movement data corresponds to movement of the body part when an object is moved by the body part.

### CROSS-REFERENCE TO PRIOR APPLICATIONS

This application claims the benefit of U.S. Provisional Application No. 62/963,737, filed on 21 Jan. 2020. This application is hereby incorporated by reference herein.

### TECHNICAL FIELD

This disclosure relates generally to processing information, and more specifically, but not exclusively, to assessing the strength of a person.

### BACKGROUND

Chronic diseases are the leading causes of morbidity and mortality in the U.S., responsible for seven out of ten deaths annually. Nearly half of adults have at least one chronic condition, contributing to 81% of hospital admissions. Among these individuals, approximately 20% are re-hospitalized within 30 days of discharge due to inadequate or poor follow-up care at home. This accounts for more than 30% of annual healthcare expenditures. Given that up to 79% of these readmissions are preventable with timely and improved outpatient care, home-based strategies designed to evaluate health status in real time have the potential to predict and detect deteriorating health, thus avoiding costly hospital readmissions by promoting proactive (instead of reactive) practices.

Some studies have suggested that there is an inverse correlation between hand grip strength and risk of death and cardiovascular disease. For example, an 11-pound decrease in grip strength over the course of the study was associated with a 16% higher risk of dying from any cause, a 17% higher risk of dying from heart disease, a 9% higher risk of stroke, and a 7% higher risk of heart attack. In fact, grip strength was even a better predictor than blood pressure.

Additional studies have shown that there is a correlation between muscle strength and functional performance and health outcomes in patients with neurological conditions, such as stroke and Parkinson's disease. The magnitude of force generation may be the most common measure of muscle contraction, and time-dependent properties of muscle contraction may be altered by neurological disease and are potentially important for function.

Existing techniques for measuring hand/finger grip and muscle strength require the use of specialized equipment. These techniques are expensive and inconvenient, requiring the patient to travel to medical facilities where the equipment can be administered by trained professionals. Moreover, the environment provided by a medical facility is not always conducive to obtaining the most accurate measurements. This is because strength tests are not performed on patients during their normal daily routines (e.g., at home or some other routine setting), when the most accurate strength measurements may be taken. This is especially a concern for chronically ill outpatients, who can experience deteriorating conditions quickly (e.g., in some cases over a few hours). Monitoring day-to-day health by performing patient strength assessments in real time and in familiar surroundings may therefore be of significant value.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a system for assessing strength of a body part;

FIG. 2 illustrates an embodiment of a system for assessing strength of a body part;

FIGS. 3A and 3B illustrate examples of objects moved by the body part for assessing strength;

FIG. 4 illustrates an embodiment of a method for assessing strength of a body part;

FIGS. 5A and 5B illustrate examples of a calibration operation;

FIGS. 6A and 6B illustrate examples of different degrees of assessed strength.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Example embodiments describe a system and method for assessing the strength of a body part of a patient in real-time and in familiar surroundings. The body part may be a finger, hand, arm, or another body part of a patient used to perform day-to-day or routine activities. The strength assessments may be performed in a non-clinical setting such as the home, workplace, nursing home, or another location with which the patient is familiar and in which the patient performs unspecialized activities. Performing strength assessments in such settings and during such activities allows the embodiments to be implemented in a manner conducive to obtaining the most accurate strength measurements.

In addition to the aforementioned features, the strength assessments taken during a monitoring period may be automatically performed without the intervention of trained professionals or the use of expensive equipment. The assessments may also be made in a manner transparent to (e.g., without specialized involvement of) the patient. Once the strength measurements are obtained, they may be processed to provide a real-time indication of the health of the patient, e.g., whether the condition of the patient is deteriorating or undergoing another event that may require treatment or observation by health care professionals.

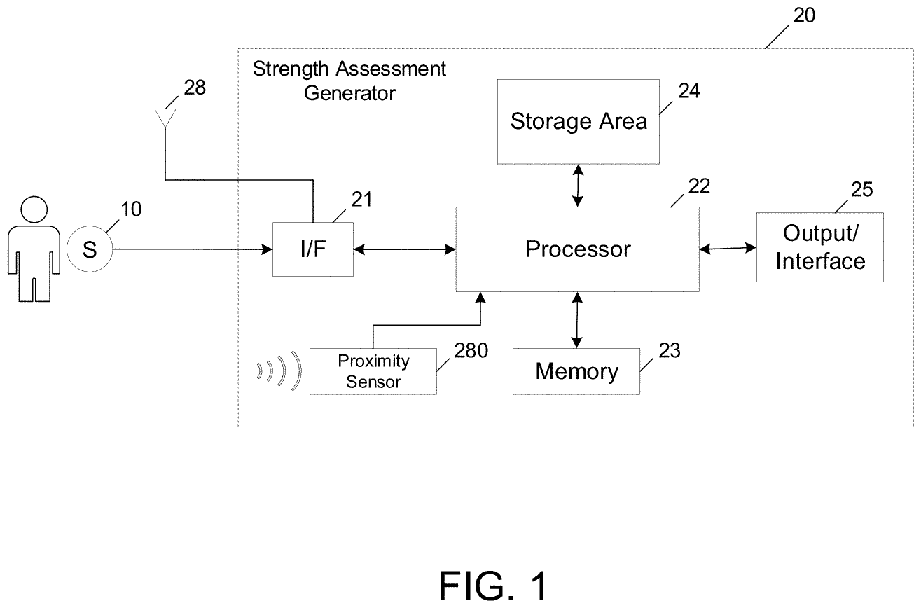

FIG. 1 illustrates an embodiment of a system for determining the health of a patient, by assessing the strength of one or more of his body parts. As previously indicated, the body parts may include a finger, a hand, or an arm. In other embodiments, the strength of another body part may be assessed, including, but not limited, to a leg, ankle, or foot of a patient. The assessed strength of the body part(s) may provide an indication of how diminished or weak the patient may be at a given time, which, in turn, may provide a basis for predicting an existing or foreseeable adverse health condition such as hospital admission, risk of fall, and frailty.

Referring to FIG. 1, the system includes at least one sensor 10 and an assessment generator 20. The at least one sensor may be worn on the body of the patient being monitored. For example, the at least one sensor 10 may be worn on or in association with a body part that is the focus of the strength assessment. The body part may be one that moves either with or independent from other parts of the body in order to perform, for example, an everyday activity. By measuring the movement of one or more body parts, sensor data may be generated that can be processed to provide a real-time indication of the relative strength of the associated body part at a given point in time. Examples of the at least one sensor 10 include, but are not limited to, one or a combination of accelerometers, gyroscopes, or other sensors that are able to measure inertial data.

The at least one sensor 10 may be incorporated into one or more devices worn or carried by the patient. The devices may include a smartphone, smartwatch, a fitness or activity monitor, or another processing device that is able to process and/or communicate signals generated by the at least one sensor or the processing of those signals for assessing strength. In a smartwatch or health watch implementation, the at least one sensor 10 may be integrated into the watch along with the assessment generator 20. In a smartphone application, the at least one sensor may communicate sensor signals to the smartphone over a wired or wireless (e.g., Bluetooth) link. In such a case, the at least one sensor may be included in a smartwatch that is linked to the smartphone of the patient.

The smartphone may include a strength assessment application executed by a processor based on instructions stored in a memory. In other embodiments, the sensor data may be transmitted to a receiver in the monitoring location where processing is performed and/or to a remote location connected to the smartphone through a network. In these cases, the assessment generator 20 may be a workstation, server, cloud-computing device, or another processing system maintained by a hospital, medical monitoring service, care provider, guardian, or at other locations. In FIG. 1, the at least one sensor 10 is illustratively shown to be connected to the assessment generator 20 through a wireless communication link 15, which, for example, may be established between a smartwatch including the at least one sensor and a smartphone including the assessment generator.

In the embodiment of FIG. 1, the assessment generator 20 may include all or a portion of an interface module 21, a processor 22, a memory 23, one or more storage areas 24, and an output or output interface 25. The interface module 21 may match the type of link 15 used to send signals from the at least one sensor 10 to the assessment generator. For example, in the case of a short-range wireless link, the interface module 21 may be a Bluetooth interface. In another embodiment, the interface module may include a plurality of interfaces, e.g., Bluetooth, WiFi, cellular, etc. For illustrative purposes, an antenna 28 is shown to transmit and/or receive information to be used in performing the strength assessment and/or communicating information relating to or controlling strength assessment operations to an external system or device, such as, for example, a server of an electronic medical records repository. In one embodiment, all the features of the aforementioned features (including the at least one sensor) may be included in a same device worn or carried by the patient. Such a device may include a smartwatch that includes both the sensor(s) and the assessment generator.

The processor 22 may be, for example, an integrated circuit, processing core, or other calculating or computing device that is capable of executing instructions stored in memory 23 for performing the operations of the strength assessment generator. The instructions may correspond to an application running on the patient device for activating or otherwise controlling the performance of strength assessments. The application may be manually initiated, for example, by the patient and/or may be automatically initiated, for example, based on one or more proximity sensors, as described in association with examples discussed below.

The storage area 24 may store medical information for the patient, sensor data, strength assessment measurements, data analytics and trend analysis information, as well as other processing results and information generated by or in association with the strength assessment application. In one embodiment, this medical information may be downloaded from an electronic medical records server and/or may be manually entered by the patient or care provider. When received from a server, the processor 22 may pre-process the data into a predetermined format compatible with the strength assessment operations controlled by the instructions in memory 23. The processing results including strength assessment information may be transmitted to a remote location (e.g., as previously described), for example, to keep up-to-date externally stored medical records of the patient.

The output/interface 25 may output the information generated by the at least one sensor and/or strength assessment generator in various forms. For example, the output 25 may include a display for indicating results of the strength assessments. Such a display may be in the device worn or carried by the patient. The output/interface 25 may generate graphics, alerts, messages, and/or other information may be generated on the display for notifying the patient. The interface 25 may include a communication circuit, for example, in the form of one of the interfaces of the interface module 21. In this case, the interface may transmit strength assessment information to a remote location for notification and/or record keeping.

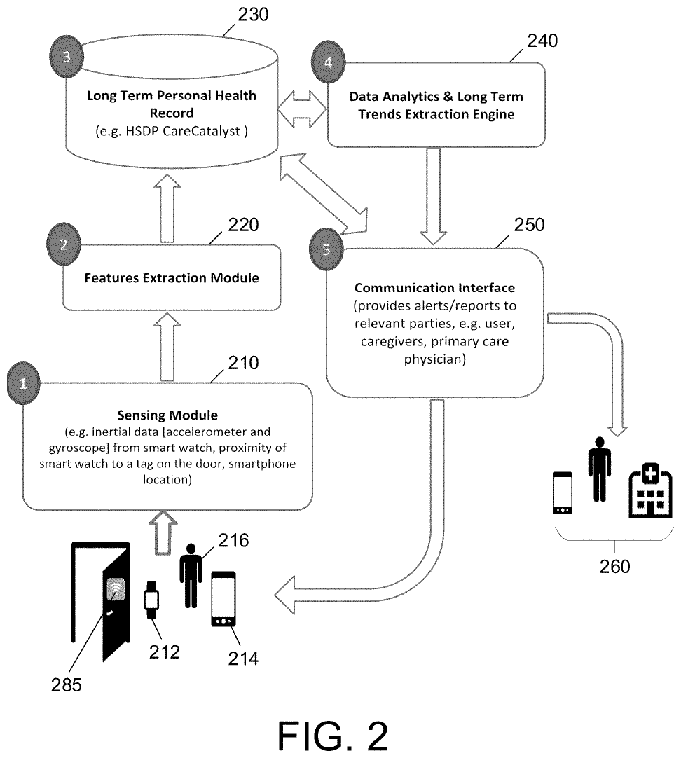

FIG. 2 illustrates an example implementation of the system of FIG. 1 for assessing the strength of a body part of a patient. In this implementation, a sensor module 210 is included in a smartwatch 212 that communicates with a smartphone 214 of the patient 216. The smartwatch is worn by the patient and the sensing module 210 generates data corresponding to a body part to be monitored. In one example, the sensor module 210 detects data corresponding to the movement of the body part(s) to which the sensor module is attached. In a smartwatch implementation, the sensor module in the smartwatch may generate data corresponding to movement of the wrist of the patient when an action is performed. The action may involve moving one or more objects in the monitoring location. According to embodiments described in greater detail below, the objects may include the handle 215 of a door 218 which is rotated, pushed, or pulled and/or the door itself, which is opened by the patient. For convenience purposes, the door may be in the home or care facility of the patient which the patient may encounter on a regular basis.

In one embodiment, the smartwatch may include a proximity sensor 280 (e.g., see FIG. 1) to detect when the patient is near (e.g., within a predetermined range or distance) the door or door handle. The proximity sensor may detect that the patient is near the door, for example, by sensing a tag 285 mounted on the door. The tag may be, for example, a radio frequency identification (RFID) tag, a near-field communications (NFC) tag, or a Bluetooth Low Energy (BLE). Detection of the tag may trigger activation of a strength assessment application in the smart watch and/or in a smartphone wirelessly linked to the smart watch. For example, when the smart watch (or smartphone) containing the proximity sensor 280 comes into the vicinity of the tag 285, a trigger signal is generated which notifies processor 22 that the patient is approaching the door and that a strength assessment is to be performed based on signals generated by sensor module 210.

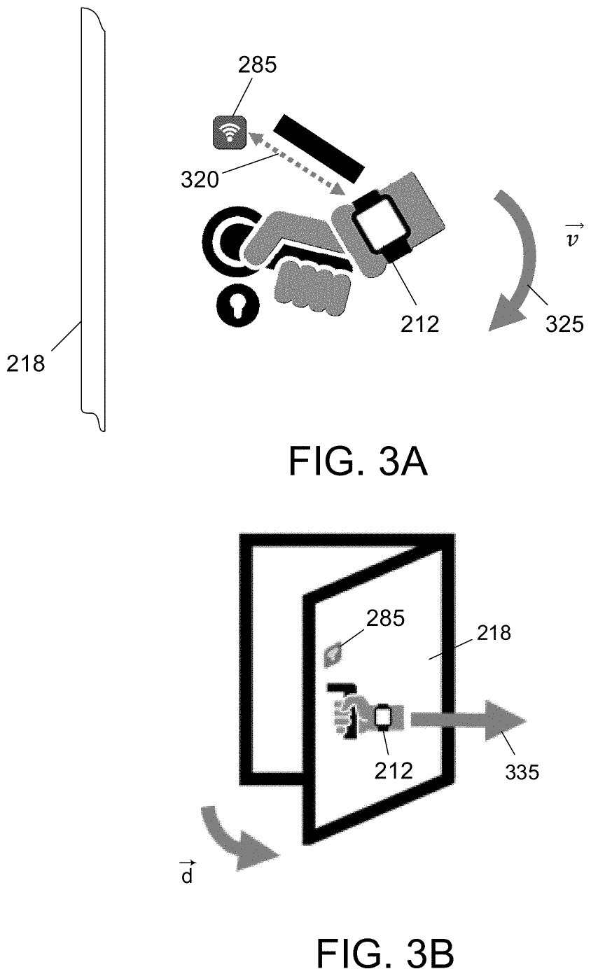

FIG. 3A illustrates an example of activating the strength assessment application when a patient rotates the handle of a door including the tag 285. In this case, the patient is wearing the smartwatch 212 including the sensor module, which, in this case, may include one or more accelerometers and/or one or more gyroscope(s) for generating data for assessing hand strength during rotation of the handle. When the patient is within range of the door, the proximity sensor 280 detects the tag 285 based on a short-range communications link 320 and then generates a trigger signal for automatically activating the strength assessment application in the smartwatch. In one embodiment, a message may be displayed on the smartwatch giving the patient the option to prevent activation of the strength assessment application, at least for this episode of rotating the handle.

Once the application is activated, the sensor module 210 generates signals based on movement of the part of the body to which the sensor module is attached, which, in this case, is the wrist. At this location, both hand strength and arm strength may be assessed. Because the patient is rotating the handle to open the door in this example, the sensor module may generate data corresponding to the angular velocity v′ (indicated by arrow 325) with which the smartwatch moved during rotation of the handle. The angular velocity data may be processed by processor 22 to provide an indication of the amount of torque that was used by the patient to turn the handle. The amount of torque may provide an indication of the hand strength of the patient, which, in turn, may serve as a real-time indicator of his health condition.

FIG. 3B illustrates an example of activating the strength assessment application when a patient opens the door (including tag 285) by rotating it about its hinges. Like in the case of turning the handle, the patient may be wearing smartwatch 212. In fact, opening the door may immediately follow turning of the handle, so that the movements in FIGS. 3A and 3B occur in succession. Once the proximity sensor 280 detects tag 285 and the application is activated, the sensor module 210 generates data indicative of the movement of the smartwatch used to open the door. This data may be indicative of the angular displacement of the smartwatch during the action of opening the door and/or another type of data. Because the patient is pulling the handle to open the door in this example, the processor 22 processes the sensor data to provide an indication of the magnitude of the pulling force (indicated by arrow 335) that was used by the patient to open the door. The magnitude of the pulling force may provide an indication of the arm strength of the patient, which, in turn, may serve as a real-time indicator of his health condition. In another embodiment, the sensor module in the smartwatch may generate data indicative of a pushing force used to push the door open. The magnitude of the pushing force may then be processed to provide an indication of arm strength.

In one embodiment, both hand strength and arm strength assessments may be taken using the same device, which in the aforementioned examples is a smartwatch. Both types of strength assessments may be taken using the same device, because of the type(s) of sensor(s) in the sensor module, e.g., accelerometers and/or gyroscopes. For example, when angular velocity or displacement is detected, the strength assessment application may determine that the angular velocity and/or angular displacement measurements are to be processed to provide an indication of forces for indicating hand and/or arm strength.

In one embodiment, the sensor(s) in the sensor module 212 may be positioned in the smartwatch along one or more predetermined axes in order to provide a basis for determining an accurate indication of the forces and/or other features used to provide the strength assessments described herein. In some embodiments, the sensor(s) are arranged to measure angular velocity and/or displacement as previously described. In such a case, accelerometers and/or gyroscopes may be positioned along multiple axes in order to capture accurate movement data. In other embodiments, a sensor may be disposed to capture movement data along just one axis.

While examples have been discussed with a device (smart watch) positioned on the wrist, the device may be positioned on another body part in other embodiments. For example, the device may be included on an arm band for measuring arm strength, on the finger for assessing finger strength, or a foot, leg, ankle, or another body part for measuring the strengths of those parts. In each of these cases, reference values may be generated or provided for the different body parts in order to provide a baseline for determining the strength assessments. Those reference values may be generated during a calibration operation, may correspond to prior or historical readings for the patient (so that real-time and previous readings may be compared to give a relative assessment of strength), and/or may correspond to objective reference values generated for patients who are similarly situated (e.g., in terms of heath condition, age, etc.) relative to the patient being monitored.

Returning to FIG. 2, in one embodiment the processor 22 may serve, at least in part, as a features extraction module 220 that processes the signals from the sensor module 210 to extract information (e.g., angular velocity, angular displacement, peak values, and/or other movement or inertial data) that may be used as a basis for assessing strength of the body part(s) of interest. The feature extraction module 220 may perform processing operations in accordance with the algorithms (e.g., equations) described in greater detail below.

The results of the strength assessments may then be sent to the storage area 230 to establish a long-term personal health record. In one embodiment, the storage area may be remotely located from the device (e.g., smartwatch) performing the strength assessment. In this case, strength assessment information may be transmitted to a remote server, database, or other device for storage. In one embodiment, the remote storage device may be a long-term health record database storing relevant information about the health of a patient. The database may be, for example, an electronic medical records (EMR) database or a tele-health database.

The system may also include an extraction engine 240 for performing operations including data analytics and long-term trend analysis based on the features extracted by the features extraction module and the strength assessments. In one embodiment, the strength assessment may be performed by the features extraction module and/or the extraction engine 240 based on the extracted features. In one implementation, the extraction engine 240 may use data from the long-term personal health record database 230 to identify trends and predict possible adverse events or health deterioration that correspond to or are based on the strength assessments generated by processor 22. The strength assessment information may be transmitted to the same or another remote server or other location for processing. Additionally, or alternatively, the data analytics and trend analysis may be performed by processor 22. In this case, processor 22 would perform the operations of the features extraction module and the extraction engine for performing the strength assessment calculations.

A communications interface 250 may communicate various types of information in response to control signals generated by the processor 22, operating as at least one of the features extraction module or the extraction engine. For example, the communications interface 250 may transmit, send, display, or otherwise provide alerts, notifications, and/or reports to one or more relevant parties 260 through text, email, phone, or other methods. The relevant parties may include one or more of the patient, informal caregivers (e.g., parent, guardian, friend, etc.), and formal caregivers (e.g., medical personnel, monitoring service, etc.).

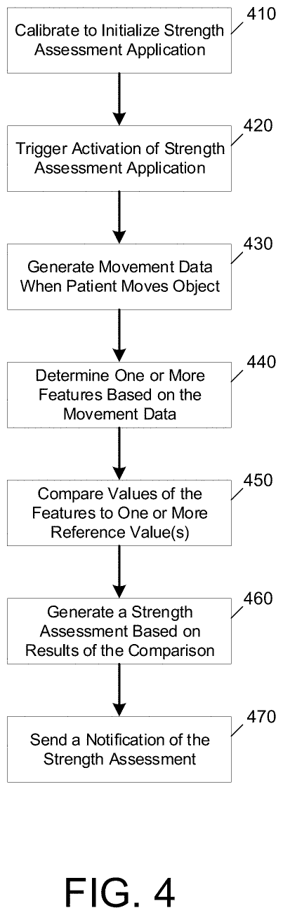

FIG. 4 illustrates an embodiment of a method for assessing the strength of a patient, and more particularly at least one body part of the patient. The method may be performed in accordance with any of the system or apparatus embodiments described herein or another system or apparatus.

Referring to FIG. 4, the method includes, at 410, performing a calibration operation to initialize the strength assessment application (e.g., the operations of the feature extraction module and/or the extraction engine performed by processor 22) in preparation for making strength assessments during a monitoring period. In one embodiment, the calibration operation may also involve generating one or more reference values to be used in establishing a baseline for performing strength assessments. In order to perform the calibration operation, first, a location is selected for the patient where the strength assessments are to be performed. The location may be any location, but for convenience may be an environment in which the patient spends considerable time performing everyday activities. Examples locations include the home or workplace of the patient. The location may also be an assisted living facility, nursing home, medical facility, or another location.

Once the location has been selected, one or more objects in that location are designated as sites for performing the strength assessments. The designated object(s) may be ones that are to be moved by a force exerted by the patient, which force or its associated values may be processed into forming the baseline reference value(s). The object may be, for example, the handle on a door, appliance (e.g., refrigerator, oven, etc.), cabinet, window, drawer, car door, or another object. The windows may have handles that are used to move the windows up and down or ones that have handles that open and close the windows by applying a torquing or turning force. The force applied by the patient may rotate the handle or may move the handle (and the object to which it is attached) along a substantially linear path as a result of a pushing or pulling force. Selection of the object may determine the body part(s) the patient is to use to apply the force, and thus the part for which strength is to be assessed. For example, as described in the examples of FIGS. 3A and 3B, the rotational force may be applied by a hand of the patient and the pushing or pulling force by the arm of the patient. These forces may be calculated in accordance with the embodiments herein to assess hand strength and arm strength.

Once the object has been selected, one or more tags are mounted in or on the object for detection by the proximity sensor in the device worn or carried by the patient. In the case where the location of interest has a plurality of objects, each to be used in performing a strength assessment of the same or different body parts, each object may include only one tag. In another embodiment, a tag may be placed on either or multiple sides or surfaces of the object to be moved by the patient.

In one embodiment, the calibration operation may involve measuring values generated when the patient moves the object one or more times. The values may be measured using the same electronic device (e.g., smartwatch) containing the sensor module, features extraction module, and other features of the embodiments described herein. The values may then be processed and stored as a baseline for the patient for the given object and movement.

In one embodiment, the patient may perform the same movement on the same object a predetermined number of times (e.g., rotating, pushing, or pulling a door handle). The sensor module in (or linked to) the electronic device worn or carried by the patient may then measure the values generated during each movement along one or more axes. In one embodiment, the values may be averaged in order to provide a more reliable measure of the present abilities of the patient. The values may be indicative of force, velocity, speed, acceleration, and/or one or more other values. The measured values may be used as a basis for generating at least one reference value, profile, or signature that serves as a custom-generated baseline to be used in assessing deviations in the strength of the patient during subsequent monitoring periods. The calibration operation may be performed for each object and for each movement of each object. In one embodiment, each of a plurality of doors at a monitoring location may be calibrated, so that strength assessments may be compared across the different doors and so that absolute forces may be measured.

As indicated above, calibration of the object to be moved by the patient (for purposes of strength assessment) may be performed in a variety of ways. In accordance with one embodiment, a calibration operation performed for a door may initially involve determining the mechanical properties of the door and door handle. The mechanical properties of the door may include the weight of the door, which may be a consideration given the fact that doors have different weights and therefore require different strengths to open them. The mechanical properties of the door handle may also be taken into consideration. These properties may include recognition of the different types of handles and thus whether a rotational, pushing, or pulling force is required for operation.

After the mechanical proprieties of the door and handle are determined, two forces may be measured. The first force (Fₕ) is the force applied by the patient to rotate the handle. The second force is the force applied by the patient to open the door (F_{d}) once the handle has been rotated into position. In one embodiment, these forces may be measured using only the sensors available in the smartwatch, which is worn on the wrist of the patient during the calibration operation. In another embodiment, the first and second forces may be determined using a dynamometer. In some implementations, the forces may correspond to, or be used to generate, the reference value(s) for performing strength assessments. (In one case, measurements for each patent may be compared relative to one another (within subject tracking) without the calibration; however, calibration may be preferred for within subject comparison for at least some embodiments.)

### Calibration and Force Calculation

An example of the calculations that may be performed by the processor 22 to calibrate the system to the door and to perform the calculations for generating one or more reference values for strength assessment will now be discussed. In this example, Equation (1) may be calculated by the processor 22 in the smartwatch containing the strength assessment application.

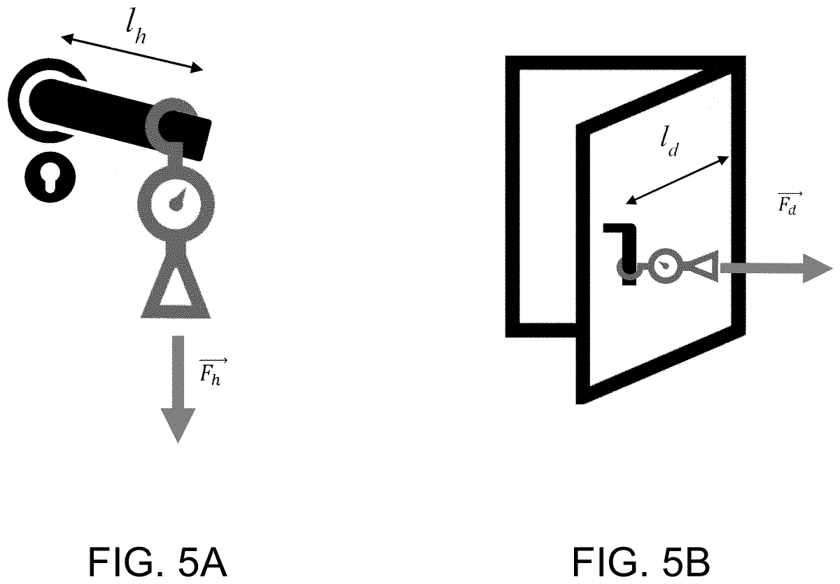

Prior to calculating Equation 1, a dynamometer 510 may be used to determine the force (Fₕ) required to rotate the handle. The product of this force Fₕ and the length of the handle lₕ (FIG. 5A) may correspond to the torque which the patient may exert in order to turn the handle into the position required to open the door.

Iᵣₕ\ddot{θ}=Fₕlₕ−Kₜθ−bₜ\dot{θ} (1)

where:
Iᵣₕ=rotational inertia of the handle
\ddot{θ}=angular acceleration of the handle
Fₕ=force applied on the handle
lₕ=length of the handle
Kₜ=torsional rigidity coefficient
θ=angular displacement of the handle
bₜ=rotary damper coefficient
\dot{θ}=angular velocity of the handle

The angular acceleration of the handle \ddot{θ} may be determined by the sensor module 210 in the smartwatch, the length of the handle lₕ is a known value, the torsional rigidity coefficient may be a known or previously calculated value, the angular displacement θ is known for the particular handle, the rotary damper coefficient bₜ may be known or previously measured for the handle, and the angular velocity \dot{θ} may be determined by the sensor module, which may include one or more accelerometers and/or one or more gyroscopes arranged along one or more predetermined axes.

In one embodiment, Equation 1 may be simplified. For example, the rotational inertia (Iᵣₕ) for the handle may be neglected because the weight of the handle is very small relative to the weight of the door. Also, it may be assumed that there is no rotary dumper element and thus its corresponding coefficient may be neglected. In view of these considerations, Equation (1) may be simplified in the manner indicated in Equation 2.

Fₕlₕ=Kₜθ (2)

During calibration, the torsional rigidity coefficient of the handle may be determined by Equation Error! Reference source not found. In this case, the force Fₕ may be determined for calibration purposes using a dynamometer (as conceptually illustrated in FIG. 5A) and lₕ is known because it is a geometrical property of the handle. The value of the angular displacement θ is measured by the sensor module of the smartwatch.

$${K}_{t} = \frac{{F}_{h} {l}_{h}}{θ} \qquad ( 3 )$$

The calibration operation performed for determining arm strength of the patient (F_{d}) may initially involve determining the force applied by the patient to open the door. In one embodiment, the arm strength F_{d} may be determined based on the equation of motion used for the door, as was performed in the case of computing hand strength. The equation of motion for the door may be given by Equation 4.

I_{rd}\ddot{φ}=F_{d}l_{d}−K_{d}φ−b_{d}\dot{φ} (4)

where:
I_{rd}=rotational inertia of the door
\ddot{φ}=angular acceleration of the door
F_{d}=force applied on the door
l_{d}=width of the door
k_{d}=stiffness coefficient of the damper
φ=angular displacement of the door
bₜ=damper coefficient of the damper
\dot{φ}=angular velocity of the door

Equation 4 may be simplified if it is taken into consideration that the door is used by the patient and that the angular velocity and acceleration of the door is negligible and thus can be neglected. The simplified version of Equation 4 may be given by Equation 5.

F_{d}l_{d}=K_{d}φ (5)

Error! Reference source not found. Error! Reference source not found. In Equation 5, the stiffness coefficient of the damper K_{d} may be determined during calibration because all the other terms of Equation 5 are known. Also, force F_{d} may be determined during calibration using the dynamometer, as conceptually illustrated in Error! Reference source not found.B. Also, the width of the door lₐ is a geometrical property of the door and is included as one of the known mechanical properties measured during calibration. Thus, the sensor module in the smartwatch may determine the angular displacement of the door φ for purposes of assessing arm strength of the patient.

Once the value of K_{d} is determined, the dynamometer may not be used unless the door is recalibrated or another door (or object) for assessing strength is calibrated. In one embodiment, once calibration has been performed, then strength assessments may be performed based on the sensing operations and data derived from the sensor module 210.

At 420, with the calibration operation complete, real-time strength assessments for determining the health of the patient may now be performed during the monitoring period. This may initially involve the proximity sensor in the device (smartwatch) detecting a tag associated with the object to be moved by the patient. Detection of the tag triggers activation of the strength assessment application in the device (or in a device or system linked to the device including the sensor module). The application may not be triggered at other times in order to prevent assessments from being performed for movements which have not been calibrated.

At 430, the sensor module in the device generates data as the patient moves the object using the body part to which the device is coupled. For example, in the case where the object is the handle of a door, the sensor module generates data based on movement of the hand of the patient (that includes the smartwatch) used to rotate the door handle. The movement data may include one or more of the features extracted by the features extraction module, as discussed herein. In one embodiment, the movement data may include angular velocity information corresponding to movement of the handle. In this or another embodiment, the movement data may include angular displacement information corresponding to movement of the door. In these or other embodiments, the movement data may correspond to different features relating to movement of the handle, door, or another object moved by the patient during the monitoring period.

At 440, the features extraction module of the device performs at least two functions. First, the features extraction module detects opening of the door based on an output of the proximity sensor. Second, the features extraction module determines one or more features based on the movement data received from the sensor module. The one or more features may include one or more of the forces Fₕ and F_{d} previously discussed, either alone or with other features to be discussed in greater detail below. For example, the values may be indicative of wrist acceleration along one, two, or three axes during a linear or torquing motion experienced by the device as the patient opens a door. In the case where the handle is rotated, the values may be indicative of wrist angular velocity.

In one embodiment, both the hand strength Fₕ and the arm strength F_{d} of the patient may be calculated for performing a strength assessment. When both forces are calculated, the arm strength of the patient may be calculated after the hand strength Fₕ has been assessed or independently from or without calculating the hand strength. In other embodiments, the hand strength or the arm strength may be determined, but not both, for performing a strength assessment.

In the monitoring period (e.g., during normal operation), the strength assessment application in the smartwatch may determine the force Fₕ which the patient applied to the handle based on the angular displacement value θ indicated by the data received from the sensor module of the smartwatch. Once this angular displacement value is received, force Fₕ may be determined using Equation 6, as the values of the other parameters Kₜ and lₕ in this equation were determined during calibration and are stored in the memory of the smart watch.

$${F}_{h} = \frac{{K}_{t} θ}{{l}_{h}} \qquad ( 6 )$$

In the monitoring period, the strength assessment application in the smartwatch may determine the force F_{d} which the patient applied to the door based on the angular displacement value φ indicated by the data received from the sensor module of the smart watch. Once this angular displacement value φ is received, force F_{d} may be determined using Equation 7, as the values of the other parameters K_{d} and lₐ in this equation were determined during calibration and are stored in the memory of the smart watch.

$${F}_{d} = \frac{{K}_{d} ϕ}{{l}_{d}} \qquad ( 4 )$$

Once one or both of these forces are calculated, a hand and/or arm strength assessment may be performed based on the output of the sensor module of the smart watch (or other patient device) including the strength assessment application.

In some embodiments, one or more additional features may be extracted by the features extraction module. For example, in addition to the forces Fₕ and F_{d}, an angular velocity waveform generated and recorded by the sensing module may be used for feature extraction. Examples of graphs corresponding to angular velocity for turning the handle of the door for both weak and strong arm strengths, with corresponding peak values a1 and a2, are illustrated in Error! Reference source not found. A and 6B, respectively. These graphs also show extracted features including time to peak values tp1 and tp2, slope of changes in angular velocity a1/tp1 and a2/tp2, and smoothness in changes in angular velocity, which, for example, may be indicated by the number of changes in slope during the patient monitoring period. As is evident by a comparison of the graphs in Error! Reference source not found.A and 6B, the changes in angular velocity are smoother for the strong arm case than the weak arm case, e.g., one change in slope compared to eleven changes. In other embodiments, a different additional set of features may be extracted by the features extraction module based on the data from the sensor module in the smart watch.
Angular velocity peak (a1 and a2)
Time to peak (tp1 and tp2)
Slope of changes in angular velocity (a1/tp1 and a2/tp2)
Smoothness in changes in angular velocity (number of changes in slope during measurement period).

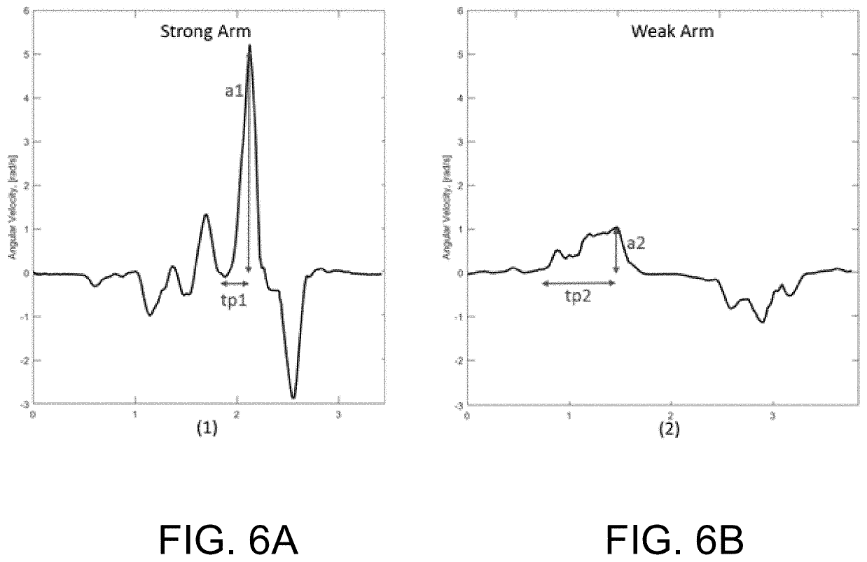

At 450, one or more of the features extracted in operation 440 may be compared to a corresponding reference values. The reference values may be ones generated during the calibration operation, ones corresponding to previous strength assessments generated for the patient and stored, for example, in memory or the electronic patient medical records, and/or generic reference values, for example, generated based on the age, health conditions, or other data relating to the patient. FIG. 6B shows an example of a reference strength assessment (e.g., waveform or profile) and FIG. 6A shows an example of a strength assessment when the patient is weak. In this case, the waveform (or one or more of its values) in FIG. 6A taken during the monitoring period may be compared with the waveform (or one or more of its values) in FIG. 6B corresponding to a reference information.

In one embodiment, one or more values in these waveforms (e.g., forces, angular velocities, angular displacements, angular velocity peaks (a1 and a2), time to peak values (tp1 and tp2), slope of changes in angular velocity (a1/tp1 and a2/tp2), smoothness in changes in angular velocity, etc.) may be subtracted from one another to generate a delta value. The delta value(s) may then be compared by the processor 22 (e.g., features extraction module, extraction engine, etc.) to one or more threshold values or predetermined ranges in order to assess whether the patient is exhibiting weak or normal strength in the body part being assessed.

For example, in one embodiment, values corresponding to one or more extracted features may be subtracted from corresponding ones of the reference values to generate delta value(s). In one embodiment, a delta value may be generated for each axis subject to the measurement or a single delta value may be generated, for example, based on a single-axis measurement or by combining the delta values along different axes to generate an aggregate delta value. In other embodiments, a waveform or profile may be generated, for example, as illustrated in FIGS. 6A and 6B in order to determine whether the patient has experienced a deterioration in strength.

At 460, a strength assessment is determined based on results of the comparison. For example, the delta value(s) may be compared to one or more predetermined ranges. In a multiple-range embodiment, the first range may include delta values considered to fall into a normal range that corresponds to no substantive deterioration in strength. The first range may include, for example, delta values ranging from 0% to 10% less than than the corresponding reference value. A second range may include delta values considered to fall into a warning range where strength is less than normal. The second range may include, for example, delta values ranging from 11% to 20% less than the corresponding reference value. A third range may include delta values considered to fall into an acute range where strength is significantly less than normal. The third range may include, for example, delta values greater than 20%. In another embodiment, the waveforms or profiles generated for the patient during the monitoring period may be compared with previous waveforms or profiles generated for the patient.

In one embodiment, the strength assessments generated in operation 460 may be input into the extraction engine to perform data analystics and a long-term trend analysis. In this case, the extraction engine 240 may retrieve data from the long-term medical database 230 to identify trends and predict possible adverse results. Examples of the information stored in the long-term medical database include relevant data about hand and/or arm strength for use in performing long-term monitoring and deterioration detection. The long-term medical database may also store results of the data analytics and trend analysis performed by the extraction engine, as well as historical data. This may provide improved personalisation for purposes of performing customized strength assessments for each patient. The database may also store other types of medical information relating to the patient that may help interpret the values extracted from the raw data for purposes of interpreting the strength assessments. Examples of these other types of medical information include, but are not limited to, demographic data and information on various health conditions or diseases that bear a relationship to diminished strength measurements.

In one embodiment, the strength assessments and/or data analystics and trend analysis may be performed by a neural network model that is generated trained (e.g., based on calibration data) to predict morbidity and/or mortality or to track movement in arm or hand performance and muscle strength. Such a model may be generated on a patient-by-patient basis and/or on a condition-by-condition basis. The conditions may include, for example, patients in different age ranges or patients with different types of health conditions or diseases. In one example implementation, the model may be generated for patients who have suffered a stroke. Because a stroke affects neurological capability, the strength assessments performed by the embodiments described herein may provide an indication of a deterioration in the health of such a patient, or even predict or detect the onset of another stroke. Other diseases or health conditions that bear a relationship with hand or arm strength include Alzheimer's disease, chronic obstructive pulmonary disease (COPD), epilepsy or other types of seizure-related diseases, and various types of neuromuscular disorders, frailty, to name a few.

At 470, once the strength assessment has been performed, the method may also include generating a message, report, alert, or other type of notification indicative of the strength assessment may be communicated to one or more parties. The reports may be sent directly to a device of the one or more parties and/or a web page of a relevant party, in addition to being transmitted to the medical database for updating the medical records of the patient. In one embodiment, the relevant parties may have an application on his or her smartphone which can be opened to determine the status of the patient based on the strength assessments. Information of the strength assessment may also be displayed on the patient device.

In accordance with one embodiment, a non-transitory computer-readable medium may store instructions that cause a processor to perform the operations of the system, apparatus, and method embodiments described herein. The computer-readable medium may correspond, for example, to memory 23 in FIG. 1, which may be any type of storage device including, but not limited to, a read-only memory, a random access memory, a flash memory, or another storage device that stores application code, firmware, software, or other types of instructions for controlling the process 22 for performing the operations described herein.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The modules, models, processors, engines, and other information generating and processing, and calculating features of the embodiments disclosed herein may be implemented in logic which, for example, may include hardware, software, or both. When implemented at least partially in hardware, the modules, models, processors, engines, calculators, and other information generating and processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, models, processors, engines, calculators, and other information generating and processing features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

Although the various exemplary embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. The embodiments may be combined to form new embodiments. Accordingly, the foregoing disclosure, description, and figures are for illustrative purposes only and do not in any way limit the invention, which is defined only by the claims.

We claim:

1. A strength assessment generator, comprising:
a memory configured to store instructions;
at least one sensor configured to detect movement data; and
a processor configured to generate a strength assessment based on the movement data detected by the at least one sensor, wherein the at least one sensor is coupled to a body part of a person and wherein the movement data corresponds to movement of the body part when an object is moved by the body part.

2. The strength assessment generator of claim 1, wherein the processor is to:
generate at least one value based on the movement data,
compare the at least one value to reference information, and
generate the strength assessment based on a result of the comparison.

3. The strength assessment generator of claim 2, wherein the reference information includes at least one of a previous strength measurement of the person, a strength measurement generated during a calibration operation, or a strength measurement corresponding to other persons with a similar health condition or demographic.

4. The strength assessment generator of claim 2, wherein the processor is to generate a strength assessment indicating deterioration in health of the person based on the comparison.

5. The strength assessment generator of claim 2, wherein:
the movement data includes an angular velocity value,
the at least one value is indicative of a force applied to the object that corresponds to the angular velocity value, and
the reference information is indicative of a reference force.

6. The strength assessment generator of claim 2, wherein:
the movement data includes an angular displacement value,
the at least one value is indicative of a force applied to the object that corresponds to the angular displacement value, and
the reference information is indicative of a reference force.

7. The strength assessment generator of claim 1, wherein the body part is a wrist of a person.

8. The strength assessment generator of claim 7, wherein:
the object is a handle of a door,
the movement data corresponds to movement of the door handle, and
the strength assessment includes assessment of hand strength.

9. The strength assessment generator of claim 7, wherein:
the object is a door,
the movement data corresponds to movement of the door, and
the strength assessment includes assessment of arm strength.

10. The strength assessment generator of claim 1, wherein the at least one sensor is included in a smartwatch coupled to the body part of the person.

11. A method for assessing strength of a patient, comprising:
detecting movement data of a person;
determining at least one value based on the movement data; and
generating a strength assessment based on the at last one value, wherein the movement data is received from at least one sensor coupled to a body part of the person and wherein the movement data corresponds to movement of the body part when an object is moved by the body part.

12. The method of claim 1, further comprising:
comparing the at least one value to reference information,
wherein the strength assessment is generated based on the comparison.

13. The method of claim 12, wherein the reference information includes at least one of a previous strength measurement of the person, a strength measurement generated during a calibration operation, or a strength measurement corresponding to other persons with a similar health condition or demographic.

14. The method of claim 12, wherein the strength assessment indicates deterioration in health of the person based on the comparison.

15. The method of claim 12, wherein:
the movement data includes an angular velocity value,
the at least one value is indicative of a force applied to the object that corresponds to the angular velocity value, and
the reference information is indicative of a reference force.

16. The method of claim 12, wherein:
the movement data includes an angular displacement value,
the at least one value is indicative of a force applied to the object that corresponds to the angular displacement value, and
the reference information is indicative of a reference force.

17. The method of claim 11, wherein the body part is a wrist of a person.

18. The method of claim 17, wherein:
the object is a handle of a door,
the movement data corresponds to movement of the door handle, and
the strength assessment includes assessment of hand strength.

19. The method of claim 17, wherein:
the object is a door,
the movement data corresponds to movement of the door, and
the strength assessment includes assessment of arm strength.

20. The method of claim 1, wherein the at least one sensor is included in a smartwatch coupled to the body part of the person.
